(19) United States
(12) Patent Application Publication
(10) Pub. No.: US 2008/0206340 A1
(43) Pub. Date: Aug. 28, 2008

(54) Galactomannans and/or Glucomannans For Increasing the Bioavailability of Active Substances

(75) Inventor: Andreas Hefel, Berlingen (CH)

Correspondence Address: BAKER & DANIELS LLP;111 E. WAYNE STREET, SUITE 800, FORT WAYNE, IN, 46802, US

(73) Assignee: Wheli Inter AG, Zug (CH)

(21) Appl. No.: 10/595,684

(22) PCT Filed: Feb. 16, 2005
(86) PCT No.: PCT/EP2005/001546

§ 371 Date: May 4, 2006

(30) Foreign Application Priority Data
Mar. 17, 2004 (DE) 10 2004 008 017.8

(51) Int. Cl. A61K 9/14; A61K 31/715; A61P 43/00

(52) U.S. Cl. 424/488; 514/54

(57) ABSTRACT

The invention relates to a method for increasing the bioavailability of nutrients by using polysaccharides such as galactomannans and similar for introducing active substances, e.g. the human growth hormone HGH and others, into the human or animal metabolism. The aim of the invention is to further develop the production of polysaccharides such as galactomannans and glucomannans in such a way that the same are also suitable for introducing active substances such as the human growth hormone into the human or animal metabolism.

U.S. Pat. No. 4,675,312 discloses the production of polysaccharide agglomerates with the object of making possible a better ingestion by avoiding the other problems of galactomannan flour, such as viscosity and adhesiveness.

Production takes place by means of two different substances, namely first by means of the galactomannan and the secondly by means of agglomeration producers that are separate therefrom.

The agglomeration producer is hardly limited in the selection of the available substances. It is only defined as a water supplier and can be of animal and/or plant origin. The percentages of the agglomeration agent in the total granulate range from 5 to 40%. Examples of such agglomeration agents are potatoes, milk and fruits.

U.S. Pat. No. 4,675,312 therefore describes the production of a granule out of galactomannan and its associated agglomeration agents.

In the US-PS only the use of these granulates as roughage is described. The finished mix is taken with fluid which with the intestinal fluid contributes to the swelling of the product. The health benefit thus is restricted to the roughage percentage that is added as a result.

The publication does not disclose how one uses such granules for the embedding of active substances. In particular the introduction of the growth hormone HGH in human or animal bodies cannot be inferred The HGH consists of a total of 188 amino acids and can only be inwardly transferred with difficulty into the human or animal body as a long-chain peptide.

The aim of the invention is thus to further develop the production of polysaccharides such as galactomannans and glucomannans specified in U.S. Pat. No. 4,675,312 in such a way that they are also suitable for introducing active substances such as for example the human growth hormone into the human or animal metabolism.

For the solution of the object the invention is characterized by the technical theory of Claim 1.

The use of granules for oral ingestion by human being and animal is described. A novel resorption kinetics of water-soluble vital substances such as HGH is claimed. The delay of the penetration of water into the granule is an advantage with regard to the retarded release of water-soluble vital substances. Fat-soluble vital substances are administered in oily suspension, as a result of which the resorption becomes nutrition independent.

The invention describes the possibility of individual combination of the described granules with their effect on the human organism.

The invention has the following features:

Use of plant ingredients

Carrier through polysaccharide

Application in various fields (anti-aging, competitive sports)

The active substances are embedded individually or as a complex separately in a vegetable matrix (Polysaccharide/guar)

The advantage is the delayed, retarded release of the active substances in the blood, the exclusion of undesirable interactions of various active substances with each other (antagonism) and the build-up of large-area resorption surfaces in the small intestine.

By means of the production of monopreparations and complexes as semi-finished preparations it is possible to manufacture completely individual vital substance preparations for human beings and animals in the simplest way.

The combination of a component system for the simple production of individual preparations and the special embedding of vital substances in plant-based polysaccharides (e.g. guar) is claimed among other things as essential to the invention.

In the following table along with the active substance HGH specified under Number 1, a number of additional active substances are listed which are supposed to be introduced with the HGH into the human or animal body. Consequently any random active substance combination of Substance 1 with all further Substances 2 through 15 is claimed as being essential to the invention.

TABLE 1
The combination of HGH with additional active substances

| | |
|---|---|
| 1. HGH - (Somatotropin) | |
| 2. L-Methionine | In the body it is transformed into |
| | cysteine, which itself is integrated to |
| | glutathione |
| 3. L-Glutathion (GSH) | Antioxidant, antitoxin and enzyme |
| | cofactor. An anti-degenerative systemic |
| | protectant. |
| 4. N-Acetyl-L-Cysteine (NAC) | A more stable form of L-cysteine. NAC |
| | is the most effective means of |
| | increasing the glutathione level |
| | in the body. |
| 5. Arginine Pyroglutamate | Triggers the release of growth |
| | hormones and enhances the |
| | perceptive function. |
| 6. Lycopene, Carotene | The most effective antioxidant. |
| (Carotenoids) | |
| 7. NADH (Nicotinamide, | Is required for the regeneration of |
| Adenine, Dinucleotide) | glutathione after it |
| | a coenzyme is oxidized. |
| 8. Alpha-Lipoic Acid (ALA) | Anitoxidant which counteracts |
| | the free radicals in the mitochondria, |
| | where the cell energy has its origin. |
| | Reduces the risk of macular |
| | degeneration. |
| 9. Chromium | Components of the glucose |
| | tolerance factor, which helps in the |
| | reduction of the blood glucose level. |
| 10. Acetyl-L-Carnitine (ALC) | reduces the dying of brain cells. |
| 11. Ginseng extract | |
| 12. Extract from green tea | well known and possesses documented |
| | anti-aging effects. |
| 13. Guar from guar gum | Galactomannan, acts as a roughage and |
| | plant-based carrier matrix of the active |
| | substances. |
| 14. Konjac from the Konjac | Glucomannan, acts as a roughage and |
| plant | plant-based carrier matrix of the active |
| | substances. |

In the following those substances which can be important for the metabolism will be designated as active substances or vital substances. Active substances can be vitamins, mineral substances, trace elements, plant ingredients, amino acids, coenzymes and other metabolic active substances of Table 1. However, the invention is not restricted solely to the active substances listed in Table 1.

The active substance is dissolved in water or in the case of fat-soluble active substances; the active substance is suspended in water. This solution or suspension is slowly introduced into the purified polysaccharide and blended. The resulting gel is dried by means of a gentle method in order not to destroy the sometimes sensitive active substances through temperature or oxygen.

The cake resulting from the drying is ground and sifted to the desired particle size (preferably 0.2-2 mm). The granule obtained in this manner has a residual moisture of about 5-7% and is thus microbiologically stable.

Upon ingestion of the granule it begins to swell and the embedded active substances are slowly released for resorption by the human or animal digestive system. A gel forms. The high compaction of the polysaccharide matrix ensures that the swelling process occurs first in the intestinal tract. During the swelling process water is continuously imbibed and therewith the matrix is loosened. Within the course of this loosening the embedded active substances can diffuse from the matrix and consequently be reabsorbed. The quantity of active substances reaching resorption hence does not exceed physiological concentrations, as can happen in the case of the release of active substances of a capsule or conventional administrative forms.

The continuous dissolution of the polysaccharide gel by means of the digestive process causes the delayed release of the embedded active substances. As a result of this behavior far-reaching concordance with the natural proportions in the consumption of vitamins or other active substances is achieved. Fruit, vegetables, meat, grains are colloidal systems, as is also the hydrocolloid galactomannan or glucomannan.

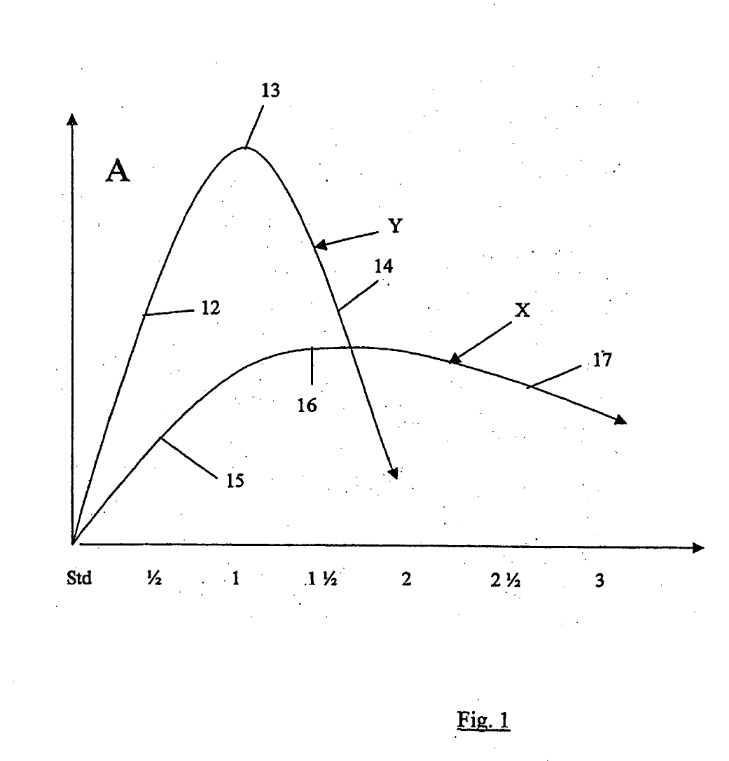

The bioavailability of the embedded active substances is thereby increased. Through the ingestion of capsules, tablets or powder practiced up to now according to the state of the art the active substance achieved high concentrations in the blood in a manner that is unphysiologically rapid and therefore is also more rapidly secreted or sometimes not even absorbed. A delay of the release of the active substance can be achieved by means of the described incorporation. The resorption kinetics that can be achieved by means of the incorporation of the active substance in polysaccharide is shown in FIG. 1.

### EXAMPLE 1

### Production of a Granule with Active Substance Coenzyme Q10

62 kg of guar meal is placed in a mixer, then a solution made of 18 kg coenzyme Q10 and 18 kg D,L-alpha tocopherol acetate is added to 15 kg of isopropyl alcohol as an antioxidant. It is mixed end then water is added until the product has achieved maximum moisture. Through the addition of the water the polysaccharide matrix begins to swell and the active substance coenzyme Q10 penetrates the polysaccharide chains and is thus immobilized. By means of subsequent drying under vacuum conditions the moisture is removed from the product at room temperature up to a residual moisture content of 5-7% and the product is consequently stabilized. The cake that is formed during the drying is broken and brought to the desired particle size of 0.2 to 2 mm by means of sifting.

### EXAMPLE 2

### Production of a Vitamin C Granule

10 kg of ascorbic acid in dissolved into 50 l of water. 30 kg of guar meal and 30 kg of konjac meal are placed in a mixer and the ascorbic acid solution is added. During the mixing the moisture content is set to the maximum achievable moisture by adding water. The mixed mass is deep-frozen, ground and then dried by means of freeze drying. The cake that is formed during the drying is broken and brought to the desired particle size of 0.2 to 2 mm by means of sifting.

### EXAMPLE 3

### Production of a Trace Element Granule

Production of a solution of 480 g of copper sulfate in 10 l of water, a second solution of 3.2 g of zinc sulfate heptahydrate in 10 l of water and a third solution of 5 g sodium selenite pentahydrate in 5 l of water. 22 kg of guar and 7 kg of potato starch are placed in a mixer and mixed. After that the individual solutions are added successively and incorporated. The maximum achievable moisture is set with water. By means of subsequent drying in a hot air stream the moisture is removed from the product up to a residual moisture content of 5-7%. The cake that is formed during the drying is broken and brought to the desired particle size of 0.2 to 2 mm by means of sifting.

The following features are therefore claimed as being essential to the invention:
Retardant effect of the incorporated active substances
Prevention of undesirable interactions between the substances, both in the preparation and in the gastrointestinal tract
Release behavior of the carrier substance (water-soluble, indigestible polysaccharide) that is close to nature, as a result improvement of the resorption properties
Improved resorption properties through the build-up of a large resorption surface in the small intestine

The subject matter of the present invention results not only from the subject matter of the individual patent claims, but rather also from the combination of the individual patent claims with one another.

All disclosed information and features, including the information and features disclosed in the abstract, in particular the spatial development shown in the drawings are claimed as being essential to the invention, in so far as they are novel compared to the state of the art individually or in combination.

In the following the invention will be described in greater detail with the help of drawings showing several embodiments. In this connection further features and advantages of the invention that are essential to the invention arise from the drawings and their description.

The figures show the following:

FIG. 1: Comparison of the kinetics of the release of the active substance in a conventional preparation compared to the active substance being incorporated in a polysaccharide;

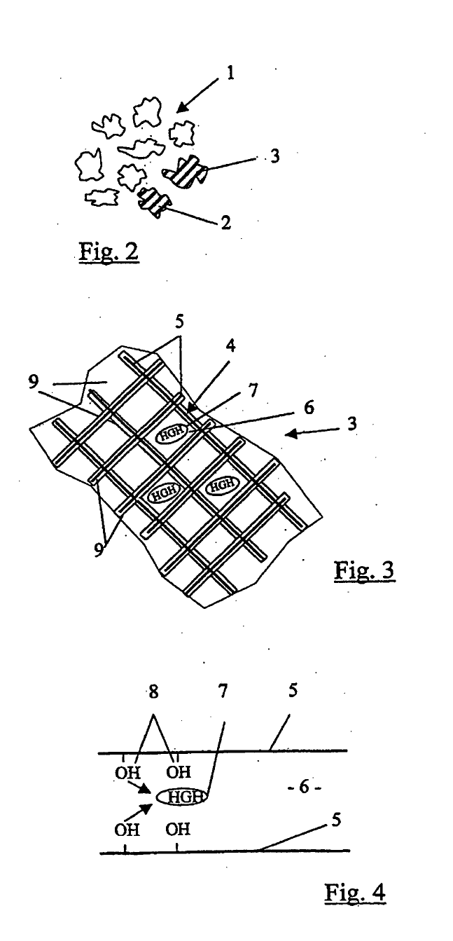

FIG. 2: an enlarged, schematic representation of a granule consisting of individual granular particles;

FIG. 3: an enlarged and schematic representation of a granular particle with the inclusion of HGH complexes;

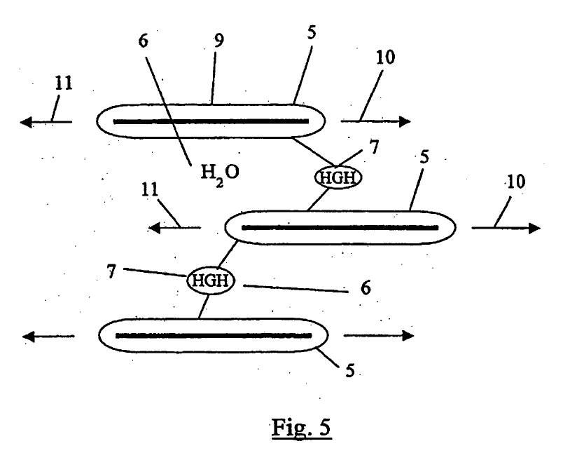

FIG. 4: a schematic representation even further enlarged compared to FIG. 3;

FIG. 5: the function kinetics of the molecular structure in the penetration of water.

FIG. 1 shows a comparison of the release of active substances in the human or animal body via two different active substance mechanisms.

The concentration of active substances in blood is shown on the ordinate while the time is shown on the abscissa.

The Y curve represents a conventional transition of an active substance into the human or animal body. As a result of this a somewhat parabolic course comes into being, i.e. a very strong increase of the concentration of active substances on the branch of the curve 12, which already culminates in the summit 13 after an hour and very rapidly declines in the region of the descending branch of the curve 14.

As a result of this the availability of the active substance is only available for a short time.

Further as a result of the steep branches of the curve 12, 14 and the high summit 13 lying in between them, concentrations of active substances that are unphysioligically high—in sometimes undesirable manner—occur.

The invention begins here, which with the flat running curve X represents an active substance incorporated in a polysaccharide and its transition into the blood of the human or animal body. The concentration of active substances increases over a longer period of time in the range of the branch of the curve 15, whereby there is only a weak summit 16, which proves that no undesirable high and unphysiological overdosages are to be feared. The decline in active substances in the range of the branch of the curve 17 is also only very slight, so that the diagram as per FIG. 1 results in the relatively high concentration of active substances at the summit 16 being retained over a very long period of time.

Hence from the comparison of Curve Y to Curve X it follows that thanks to the technical measures of the invention a high concentration of active substances in the blood can be achieved over a longer period of time.

The graphic illustrates the possibility of a desired resorption delay by means of the embedding of the active substance in a polysaccharide. This means a uniform supply and a better use of the active substances in the human and/or animal metabolism.

FIG. 2 shows a granule 1 as an example which consists of a multitude of granular particles 2, 3.

For example, ascorbic acid is embedded in the one granular particle, as described in the aforementioned Example 2.

In the other granular particle the growth hormone HGH is for example embedded, as graphically represented as an HGH complex. This integration mechanism is mentioned in Example 3 of the foregoing description.

It is important that the two granular particles 2, 3 are completely separate in function and do not blend or come into interaction with one another in undesirable manner.

Because the active substances (ascorbic acid and selenite) are integrated in different granular particles 2, 3, an undesirable interaction between these active substances is therefore prevented in the gastrointestinal tract.

Details of the incorporation of an HGH complex 7 are described in greater detail with the assistance of FIGS. 3 through 5.

In the enlarged electron-microscopic representation of a granular particle 3 it turns out that said particle is formed from a multitude of net-shaped or lattice-like polysaccharide molecules 5, which form a lattice structure 4.

The HGH complexes 7 are now incorporated in the interstitial spaces 6 of this lattice structure 4 by means of a coordinate link to the lattice structure 4 of the polysaccharide molecules 5.

It is also to be mentioned that the polysaccharide molecules 5 themselves are enclosed by a depicted H₂O shell which completely envelops and screens the linear structure.

In the further enlarged display as per FIG. 4 it can be seen that OH groups are attached to the linear polysaccharide molecules 5, said OH groups being a component of the polysaccharide molecule 5.

The HGH complexes 7 are incorporated in the interstitial space 6 between the molecules 5 on the basis of the previously mentioned coordinate link. In this connection the HGH complexes are polyvalent positive, while the OH group 8 bears a negative split ionic charge.

In this way the HGH complexes are held in the interstitial space 6 between the linear polysaccharide ions on the basis of the described coordinate link.

With this the delayed release is substantiated because when the water penetrates into the as per FIG. 4 the reaction kinetics as per FIG. 5 results.

There it is in turn possible to recognize that the polysaccharide molecules 5 enclosed by a hydrational shell are bound to one another in the interstitial space by water molecules, in whose interstitial space the HGH complexes 7 are in turn also present.

If water or intestinal fluid now penetrates into the interstitial spaces 6, then there is a partial cancellation of the bond between the molecules 5, and said molecules shift towards one another two-dimensionally in the direction of the arrows 10, 11.

With this the bond between the polysaccharide molecules 5 is partially cancelled and the HGH complexes 7 are released into the surrounding fluid.

With this the delayed release is substantiated, because one more partial adhesion and bond is present in the interstitial space 6 between the polysaccharide molecules 5. Further the delayed release is substantiated by the fact that the individual threads are carried through the penetrating water or the intestinal fluid in layers and with this the lattice structure is also carried away in layers, in order to in this way release the HGH complexes 7 embedded in the interstitial space 6.

In the following describes how the previously described hydrational shell 9 came about.

In the dry meal the galactomannan fibers stick very closely together. By mixing of this network with water these threads loosen and surround themselves with the previously mentioned hydrational shell 9.

In this way it is possible to create in inventive manner the lattice structure of the polysaccharide molecules 5 in such a way that said molecules are enclosed by the mentioned hydrational shell (H₂O shell 9).

This hydrational shell provides the interlink between the individual polysaccharide molecules 5, as shown with the assistance of the reaction kinetics of FIG. 5.

### KEY TO DRAWINGS

1 Granule
2 Granular particle (Asc)
3 Granular particle (Se)
4 Lattice structure
5 Polysaccharide molecule
6 Interstitial space
7 HGH complex
8 OH group
9 Hydrational shell
10 Direction of arrow
11 Direction of arrow
12 Branch of the curve
13 Summit
14 Branch of the curve
15 Branch of the curve
16 Summit
17 Branch of the curve

1-21. (canceled)

22: Procedure for the increase of the nutrient-bio-availability of vital substances in a test person that wishes such an increase for improvement of wellbeing, which includes the administration of a nutritiously active quantity of at least one nutritional additive and a quantity of galactomannan and/or glucomannan that increases the bio-availability, characterized by that water-soluble or fat-soluble, in the water suspended, vital substances are embedded in a botanical matrix of a polysaccharide individually or as a complex separately and always separated from each other in their function, whereby the from this obtained granulate swells when taken and the embedded active substances are slowly released for resorption by the human or animal digestive system.

23: Procedure according to claim 22, characterized by that HGH (source somatotropin) is embedded in galactomannan and/or glucomannan.

24: Procedure according to claim 22, characterized by that the mentioned nutritional material comprises at least one material that is selected from a group that consists of that consists of herbal extracts, water-soluble vitamins, fat-soluble vitamins, amino acids, fatty acids, minerals and antioxidants and hormones.

25: Procedure according to claim 22, characterized by that the mentioned herbal extract is selected from a group that consists of ashwaganda, boswellin, capsaicin, curcumin, holy thistle extract, sceletium, and ayurvedic herbal extracts.

26. Procedure according to claim 22, characterized by that the mentioned water-soluble vitamins are selected from a group that consists of vitamin B1, vitamin B2, niacin, vitamin B6, vitamin B12, folacin, inositol, vitamin B5, and vitamin C, whereby the fat-soluble vitamins are selected from a group that consists of vitamin A, vitamin D, vitamin E, and biotin.

27. Procedure according to claim 22, characterized by that the mentioned water-soluble vitamins are selected from a group that consists of vitamin B1, vitamin B2, niacin, vitamin B6, vitamin B12, folacin, inositol, pantothenic acid, and vitamin C, whereby the fat-soluble vitamins are selected from a group that consists of vitamin A, vitamin D, vitamin E, and biotin.

28. Procedure according to claim 22, characterized by that the mentioned antioxidants are selected from a group that consists of mixed carotenoids, the co-enzyme Q10, lycopenes, lutein, zeaxanthin, bioflavonoids, germanium, selenium, zinc, vitamin A, vitamin C, und vitamin E, alpha-Lipoic, grape sperm phytosome, extract from green tea and extract from pine bark.

29. Procedure according to claim 22, characterized by that the mentioned amino acids are selected from a group that consists of N-acetyl-cysteine, acetyl-L carnitine, L-arginine HCL, L-carnitine, endorphenyl D-phenylalanine, GABA, L-glutamine, L-glycine, L-histidine, L-lysin, L-methinin, L- and DL-phenylalanine, proline, taurine, 5-hydroxy-tryptophan, L-tyrosin.

30. Procedure according to claim 22, characterized by that the mentioned minerals are selected from a group that consists of calcium, chrome, copper, germanium, lead, iron, magnesium, manganese, selenium, silicon, vanadium, zinc.

31. Application of polysaccharides, such as galactomannans, glucomannans, and of a similar kind, for the infiltration of active substances according to claim 22, characterized by that the vital substances are embedded in a botanical matrix individually or as a complex separately and always separated from each other in their function.

32. Application according to claim 31, characterized by that the vital substances are vitamins, minerals, trace elements, plant content substances, amino acids, coenzymes, and other metabolically active substances.

33. Application according to claim 31, characterized by that the active substance is dissolved in water or, in the case of fat-soluble active substance, is suspended in water, the solution or suspension is slowly added to and mixed with the purified polysaccharide, the emerging gel is dried by an economizing procedure, the clog that forms from the drying fragments and is filtered for the desired grain size (preferably 0.2-2 mm).

34. Polysaccharide according to claim 31, characterized by that a granulate contains a multitude of granulate particles, whereby in a first granulate particle a first active substance and in a second granulate particle a second active substance is embedded.

35. Polysaccharide according to claim 34, characterized by that the granulate particle are separated in function and do not interact with each other in an undesired way.

36. Polysaccharide according to claim 31, characterized by that the granulate particle contains a multitude of grid or grate shaped polysaccharide molecules, which form a lattice pattern, whereby in the interstices of the lattice pattern the ions of the active substance are bonded through a coordinate bond in the lattice pattern of the polysaccharide molecules.

37. Polysaccharide according to claim 31, characterized by that polysaccharide molecules contain a surrounding H2O surface film, that completely encloses and shields the thread-like structure.

38. Polysaccharide according to claim 31, characterized by that the thread-like polysaccharide molecules OH groups and that ions of the active substance in the interstice between the molecules are bonded by a coordinate bond.

39. Polysaccharide according to claim 31, characterized by that because of the penetration of water or intestinal fluids in the interstices of the molecules these move two dimensionally opposite to each other.

40. Polysaccharide according to claim 31, characterized by that the active substance exhibits a delayed release, whereby the single threads are in layers removed by the penetrating water or the intestinal fluids, whereby the grating structure is removed in layers, and releases the active substance ions that are adsorbed in the interstices.

41. Polysaccharide according to claim 31, characterized by that the thread-like molecules are surrounded by a hydrate coat.
